(19) United States
(12) Patent Application Publication
(10) Pub. No.: US 2009/0254094 A1
(43) Pub. Date: Oct. 8, 2009

(54) RATCHETING MECHANICAL DRIVER FOR CANNULATED SURGICAL SYSTEMS

(76) Inventors: Troy D. Knapp, Negaunee, MI (US); Phillip J. Berman, Negaunee, MI (US)

Correspondence Address: FITCH EVEN TABIN & FLANNERY, 120 SOUTH LASALLE STREET, SUITE 1600, CHICAGO, IL, 60603-3406, US

(21) Appl. No.: 12/420,645

(22) Filed: Apr. 8, 2009

Related U.S. Application Data
(60) Provisional application No. 61/043,206, filed on Apr. 8, 2008.

(51) Int. Cl. A61B 17/58

(52) U.S. Cl. 606/96; 606/104

(57) ABSTRACT

A device for rotatably driving a cannulated tool along a guide wire. In one aspect, the device includes a coupling assembly for receiving the tool and guide wire as well as a ratchet mechanism permitting selective rotation between the device and the tool. The device further includes an elongate gripping portion extending transversely to the coupling assembly to provide a levered mechanical advantage while positioning the gripping portion out of the path of the guide wire. In another aspect, the device includes a handle having a through bore shorter than a predetermined length of a connection assembly through bore so that only a short portion of the guide wire is covered by the device. A method of driving a cannulated tool along a guide wire is also provided that includes rotating a gripping portion of a handle assembly about the tool in an arcuate path spaced from the tool.

### CROSS-REFERENCE To RELATED APPLICATIONS

This application claims the benefit of U.S. Provisional Patent Application Ser. No. 61/043,206, filed Apr. 8, 2008, which is hereby incorporated by reference as if fully set forth herein.

### FIELD OF THE INVENTION

The invention relates to an apparatus and method for manipulating a cannulated surgical tool and, more particularly, to a device having a ratchet mechanism for selectively permitting rotation of the surgical tool relative to the device.

### BACKGROUND OF THE INVENTION

Implant devices secured to bone or bone segments are utilized to promote the healing and repair of various parts of the human body. In some cases, the implant devices are secured to the bone or bone segments such that the bones themselves heal, fuse, or stabilize relative to one another. In other cases, implant or fixation devices are used to secure bones or bone fragments so that the surrounding soft tissue may heal without disruption by relative movement of the bones.

During the surgical procedure to implant the fixation devices, a plurality of bone screws or other fixation elements are secured to a plurality of respective bones. Then, each of the bone screws is secured relative to the others with an additional apparatus, such as a connecting member or rod. The process of implanting the bone screws into the bones or bone fragments requires a high level of accuracy despite the numerous tissues within the patient's body which tend to obstruct the surgical site. In some surgeries, guide wires are used to guide surgical tools and bone screws along a predetermined path to the target bone surface.

For example, spinal rods that immobilize vertebral bones of the spinal column are typically anchored to the vertebrae via bone screws that extend through boney structures of the vertebrae called pedicles. To begin the procedure, an opening or an incision is made near the vertebrae to be immobilized. A hand-held instrument known as a Jamshidi needle is inserted through the incision and used to locate the pedicle of a vertebra along the posterior spinal anatomy of the patient. Using the Jamshidi needle, a surgeon may rely upon tactile feedback to determine the desired location for the bone screw on the pedicle. Once positioned, the Jamshidi needle is partially driven into the cancellous portion of the vertebral body in the desired trajectory for the bone screw. A guide wire is inserted through the Jamshidi needle and lightly tapped with a hammer to secure the guide wire to the vertebral bone before the Jamshidi needle is removed.

The guide wire functions to guide instruments and bones screws within the surgical field into contact with the pedicle at the desired trajectory. To provide a path to the vertebra, a series of tissue dilators with increasing diameters are passed along the guide wire and into contact with the pedicle. Once the surrounding tissue is sufficiently stretched, a docking port is positioned in the opening and functions as an access window through which the rest of the procedure is conducted.

After the docking port is secured in the opening, the surface of the vertebra may be prepared to facilitate attachment of the bone screw to the vertebra. A variety of different tools may be used, including an awl to perforate the cortex of the vertebra, a pedicle finder to create a pilot hole in the vertebra, and a tap to cut threads into the vertebra. A tool may engage the surface of the vertebra in a number of different ways, but often tools are used which rely upon rotational movement to create contact between the cutting surfaces of the tool and the bone. Further, each tool is typically cannulated so that it may be passed over the guide wire and travel therealong until it contacts the bone surface. At that point, the surgeon may rotate the tool to engage the surface of the vertebra and prepare the implant site to receive the bone screw.

It has been observed, however, that the cannula of the tool may become obstructed during use which causes the tool to bind to the guide wire. If the cannulated tool and guide wire become bound together, driving the tool into the vertebra may cause the guide wire to be driven through the vertebral body and into harmful contact with the structures of the circulatory or nervous systems. Normally, the guide wire is longer than the cannulated tool such that the guide wire projects beyond the tool during use. This permits a surgeon to monitor the length of the guide wire projecting beyond the tool and verify that the guide wire is relatively stationary and has not been driven through the vertebral body.

In some procedures, the guide wire is initially positioned further into the bone than the cannulated tool. For example, it may be desirable to drill and tap a pilot hole for the bone screw. A pedicle finder is first passed over the guide wire and into contact with the vertebra to drill the pilot hole to a predetermined depth. The guide wire would then be seated within the vertebra at the end of the pilot hole after the drilling has been completed. After removing the pedicle finder, a cannulated tap is placed over the guide wire and advanced into the pilot hole until it also reaches the end of the pilot hole. As the tap travels through the pilot hole, the length of the guide wire extending beyond the cannulated tool continues to increase until the tool reaches the predetermined depth. In this regard, the ability of the surgeon to monitor the length of the guide wire projecting beyond the tool permits verification of the progress of the tool into the bone.

Despite the benefits of using a guide wire with cannulated tools, the guide wire is often problematic in that the presence of the guide wire extending beyond the tool may restrict the user's movement. More specifically, conventional cannulated tools include a palm handle or T-handle design that positions the user's hand directly in the path of the guide wire. To compensate for the presence of the guide wire, a biomechanically awkward hand position along the gripping surface is often used. This positioning tends to limit the ability to visually monitor the length of the guide wire projecting beyond the cannulated tool. Additionally, the guide wire may potentially puncture the glove or hand of the user, especially if the end of the guide wire is sharp. These designs also require rotation of the tool through complete revolutions when engaging or disengaging the tool with the bone. Prior attempts to remedy these shortcomings fail to provide sufficient mechanical advantage and operability such that a user may quickly and efficiently engage or disengage the cannulated tool with the bone.

Accordingly, a device and method for manipulating rotatable cannulated tools that is easier to use would be desirable. More particularly, a device that positions a gripping surface out of the path of the guide wire while providing a significant mechanical advantage would be desirable. The device should permit a user to apply the substantial force necessary to engage or disengage tools or screws relative to bone that may be difficult to generate using linear tools. Further, a device that permits rotation of a cannulated tool while preserving the ability to visually monitor the position of a guide wire extending through the cannulated tool is also desirable. In addition, an easier way to rotatably engage or disengage a cannulated tool with the surface of a bone would be desirable.

### SUMMARY OF THE INVENTION

In accordance with one aspect of the invention, a device for rotatably driving a cannulated tool along a guide wire is provided that allows a user to exert a rotary force to the cannulated tool via an elongate gripping portion positioned away from the guide wire. In this regard, the device has an elongate coupling assembly with an axial through bore in which the tool and the guide wire extending through the tool are received. The device has a handle assembly for rotatably driving the tool, the handle assembly including an end portion connected to the coupling assembly. Positioned between the handle assembly and the coupling assembly is a ratchet mechanism configured so that turning the handle assembly in one rotary direction turns the coupling assembly therewith. The coupling assembly is also configured so that the handle assembly turns relative to the coupling assembly in an opposite rotary direction. Additionally, the device includes an elongate gripping portion of the handle assembly extending transversely to the coupling assembly. By grasping the handle assembly gripping portion, a user may exert a levered mechanical advantage for turning the coupling assembly and cannulated tool received therein.

In accordance with another aspect of the invention, a mechanical driver for rotatably driving a cannulated tool is provided wherein only a short portion of the guide wire is enclosed by the device so that the position of the guide wire relative to the cannulated tool may be readily determined. The device includes a coupling socket for removably receiving an end of the cannulated tool. The device also includes a transverse handle for being gripped to rotate the coupling socket and tool. The transverse handle includes upper and lower surfaces, as well as a coupling end portion having a through bore between the upper and lower surfaces of the handle. In between the socket and transverse handle, a cannulated connection assembly is positioned having an elongate through bore in communication at one end with the socket opening. The through bore of the connection assembly generally extends transversely to the upper and lower surfaces of the handle.

The connection assembly is attached to the handle coupling end portion so that the through bores of the connection assembly and the transverse handle are coaxial. The through bore of the handle coupling end portion is relatively short compared to the length of the connection assembly through bore. Specifically, the through bore of the handle coupling end portion is shorter than the connection assembly through bore as measured along the connection assembly through bore from the end in communication with the socket opening to the upper surface of the handle. Accordingly, the mechanical driver limits the length of the connection assembly through bore that overlies the guide wire extending beyond the cannulated tool. This permits the use of shorter guide wires while preserving the ability to visually monitor the position of the guide wire relative to the cannulated tool.

A method of driving a cannulated tool along a guide wire and into a bone is also provided that positions a gripping portion of a handle assembly away from the path of the guide wire. The method includes releasably connecting the handle assembly to the tool and positioning the tool adjacent the surface of the bone. The method also includes rotating the gripping portion of the handle assembly about the tool in an arcuate path spaced from the tool. In this manner, the gripping portion is positioned out of the path of the guide wire as the tool is rotatably driven into the bone.

### BRIEF DESCRIPTION OF THE DRAWINGS

FIG. 1 is a perspective view of a device in the form of a mechanical driver for manipulating rotatable cannulated surgical tools that shows a coupling assembly for attaching to the tool and an elongate handle extending transverse to the coupling assembly;

FIG. 2 is a side view of the mechanical driver of FIG. 1 showing the driver connected to a cannulated tool and positioned along a guide wire with the guide wire protruding from the driver spaced a distance from a gripping portion of the device;

FIG. 3 is a front view of the mechanical driver showing aligned through bores of the coupling assembly for receiving the tool and the smaller diameter guide wire;

FIG. 4 is a back view of the mechanical driver showing the smaller through bore of the coupling assembly extending through an end portion of the handle;

FIG. 5 is a top view showing a length between upper and lower surfaces of the handle that is less than the longitudinal length of the coupling assembly;

FIG. 6 is a side view of the mechanical driver showing features of the handle gripping portion that are complimentary to a user's hand;

FIG. 7 is a perspective view of the mechanical driver with the coupling assembly removed to show a through bore formed in the handle end portion for receiving the coupling assembly.

FIG. 8 is a cross-sectional view of the handle of FIG. 7 showing the internal geometry of the handle configured to receive an end of the coupling assembly;

FIG. 9 is a cross-sectional view of an embodiment of the mechanical driver showing the internal components of the coupling assembly;

FIG. 10 is a cross-sectional view of the mechanical driver of FIG. 9 connected to a cannulated tool and positioned along a guide wire showing a portion of the guide wire extending beyond the end of the tool within the coupling assembly;

FIG. 11 is an exploded view of an embodiment of the mechanical driver showing the assembly of the driver onto a cannulated tool and guide wire;

FIG. 12 is a side view of the assembled mechanical driver, tool, and guide wire of FIG. 11 showing a flat upper surface of the handle for striking with a mallet to initially engage the tool with a bone; and

FIG. 13 is a perspective view of the mechanical driver of FIG. 11 showing the movement of the driver and the tool during a procedure.

### DETAILED DESCRIPTION OF THE PREFERRED EMBODIMENTS

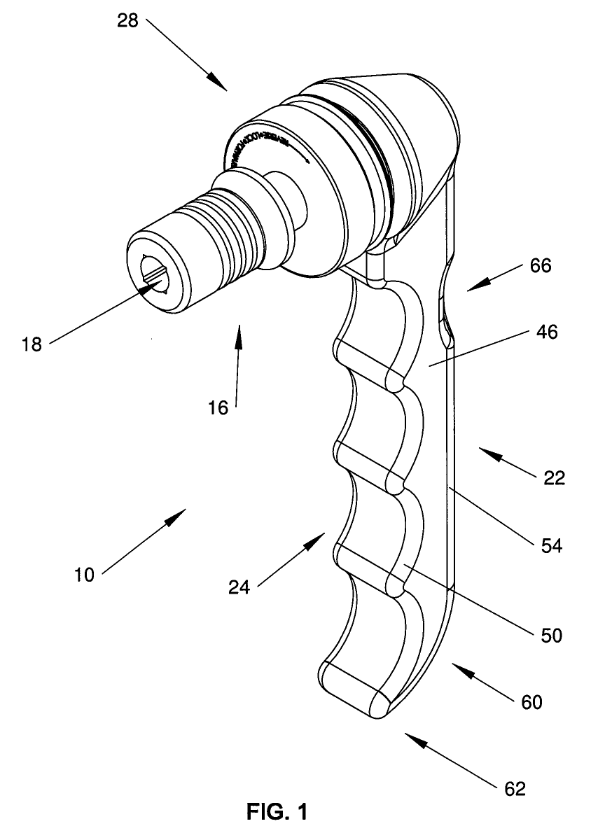

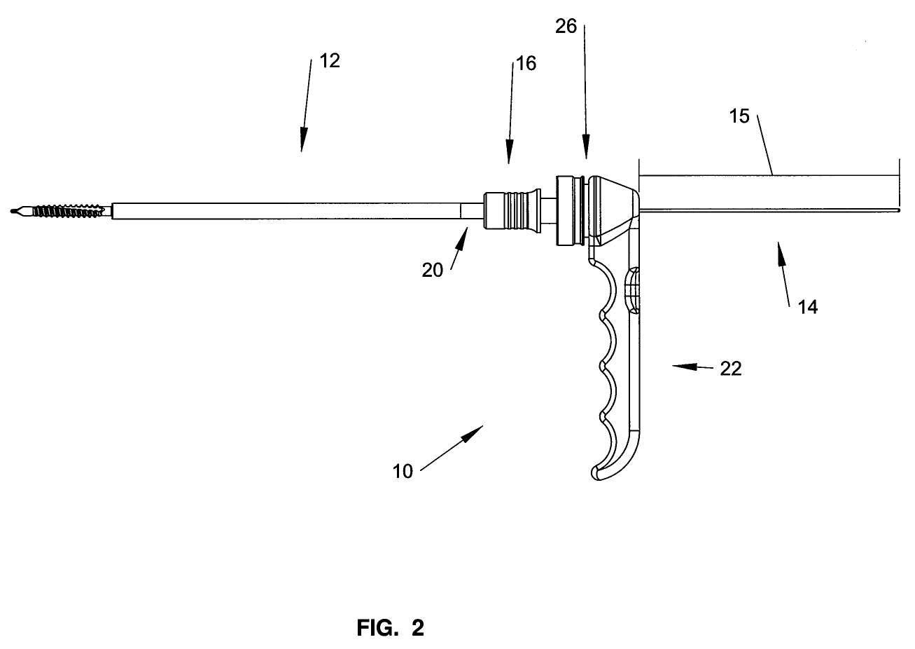

In FIGS. 1 and 2, a device in the form of a mechanical driver 10 for rotatably driving a cannulated tool 12 along a guide wire 14 is shown. The mechanical driver 10 has a coupling assembly 16 with a through bore 18 for receiving an end 20 of the cannulated tool 12 and a guide wire 14 extending therethrough. A handle assembly 22 having an elongate gripping portion 24 extends transversely to the coupling assembly 16. The coupling assembly 16 is configured to quickly and releasably engage the end 20 of a variety of different tools. Additionally, the mechanical driver 10 is configured to permit the guide wire 14 to pass through the coupling assembly 16 and beyond the handle assembly 22.

During an operation, a guide wire 14 is placed adjacent a bone surface to guide the cannulated tool 12 into contact with the bone. Then, the mechanical driver 10 is connected to the cannulated tool 12 before sliding the tool 12 along the guide wire 14. The handle assembly 22 spaces the user's hand from the path of the guide wire 14 as the tool 12 and mechanical driver 10 slide along the guide wire 14. The handle assembly 22 also provides a levered mechanical advantage to rotatably drive the tool 12 into the bone. To permit relative rotation between the handle assembly 22 and the coupling assembly 16, the coupling assembly 16 includes a ratchet mechanism 26 disposed between the handle assembly 22 and the coupling assembly 16. As is apparent, the mechanical driver 10 provides a safe and efficient way to manipulate cannulated tools positioned on a guide wire when compared to prior approaches.

During a number of procedures, it is desirable to visually monitor a portion 15 of the guide wire 14 extending beyond the handle assembly 22. For instance, to protect against driving the guide wire 14 beyond the bone, the user may monitor the portion 15 to observe whether the tool 12 has become bound with the guide wire 14. In another procedure, the user places the guide wire 14 a predetermined distance into the bone and the guide wire 14 translates relative to the tool 300 and mechanical driver 10 as the tool 300 is driven into the bone. The user continually monitors the portion 15 of the guide wire 14 projecting beyond the handle assembly 22 to verify the progress of the tool 12 into the bone. As will be discussed in greater detail below, the length of the guide wire 14 obscured by the mechanical driver 10 is generally determined by the length of the coupling assembly 16 instead of the handle assembly 22. In other words, the portion 15 is maximized for any given guide wire 14 because the gripping portion 24 extends transversely to the coupling assembly 16 instead of overlying the guide wire 14. This design allows a user to monitor the position of the guide wire 14 over a greater range of motion, as well as to use a shorter guide wire 14 while preserving the ability to observe its position.

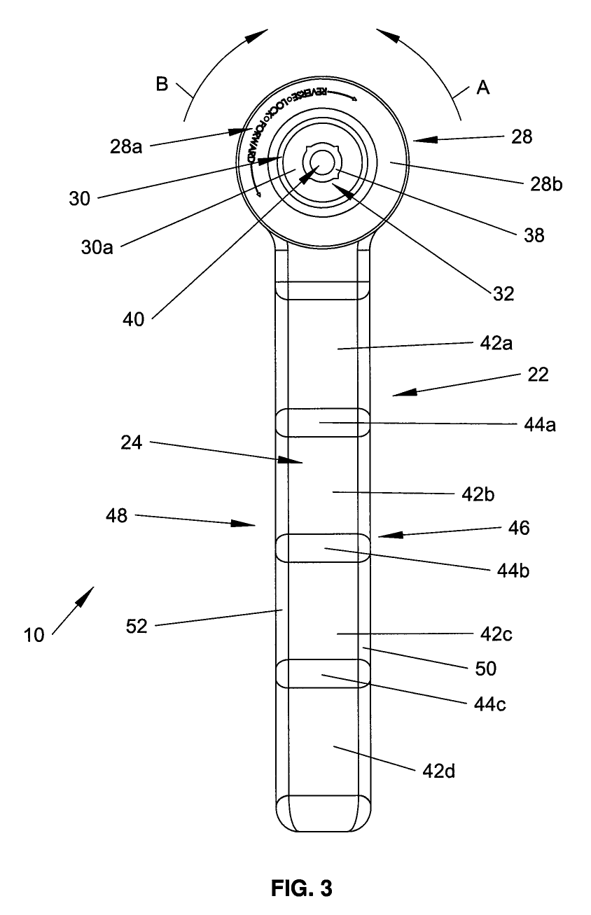

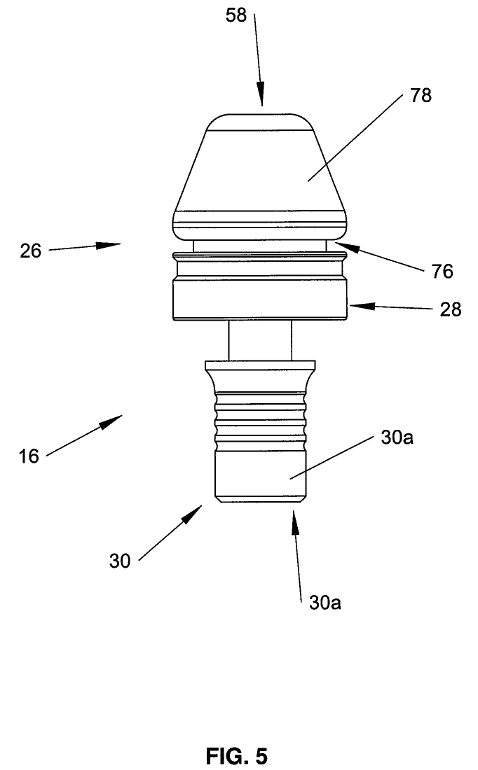

As best seen in FIGS. 3-6, the coupling assembly 16 also includes a drive reverser or rotation selector 28 and a coupling socket 30 positioned at one end of the handle assembly 22. The rotation selector 28 is rotated to selectively limit relative rotation between the coupling socket 30 and the handle assembly 22, while the coupling socket 30 is configured to releasably receive a variety of tool ends 20 within socket opening 32. In a preferred form, these components are made from stainless steel.

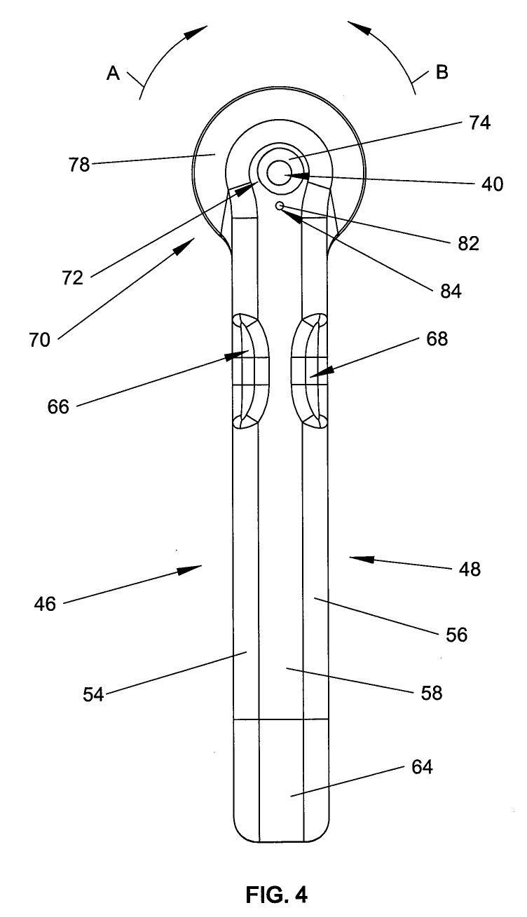

Indicia 28a may be placed on face 28b of the rotation selector 28 to aid a user in operating the mechanical driver 10. It is preferred that the indicia 28a be configured to represent the behavior of the rotatable tools that will be used with the mechanical driver 10. Stated differently, if the rotatable tools typically engage a bone surface when rotated in a clockwise direction (direction A in FIG. 4) the “forward” and corresponding arrow in indicia 28a should be configured such that a user would rotate the rotation selector 28 in the “forward” direction in order to engage the bone with the tool. Conversely, if the rotatable tools typically disengage from the bone when rotated in the counter-clockwise direction (direction B in FIG. 4) the rotation selector 28 would be rotated in the “reverse” direction in order to disengage the tool from the bone.

For example, when the rotation selector 28 is rotated in the “forward” direction indicated by arrow A, the mechanical driver 10 is configured to drive the tool 12 into a bone. More specifically, turning the handle assembly 22 in direction A turns both the coupling socket 30 and tool 12 in direction A. This drives the tool 12 deeper into the bone. However, the handle assembly 22 turns freely relative to the coupling socket 30 when the handle assembly 22 is turned in direction B. This free turning permits a user to utilize an arc of rotation of the handle assembly 22 that is less than 360 degrees without releasing the handle assembly 22 or disengaging the tool 12. Thus, after turning the handle assembly 22 through a short arc in direction A and turning the tool 12 a corresponding amount, the user turns the handle assembly 22 in direction B back to the starting point of the short arc without affecting the position of the tool 12, and then rotates the handle assembly 22 in direction A to drive the tool 12 deeper into the bone.

By contrast, rotating the rotation selector 28 in the “reverse” direction indicated by arrow B configures the mechanical driver 10 to disengage the tool 12 from the bone by reversing the operation of ratchet mechanism 26. Once the tool 12 has reached the target depth within the bone, the rotation selector 28 is rotated in direction B to fix handle assembly 22, coupling socket 30, and tool 12 against relative rotation when the handle is turned in direction B. Thus, turning the handle assembly 22 in direction B tends to reverse the path of the tool 12 within the bone and eventually removes the tool 12 from the bone. However, when the handle assembly 22 is turned in direction A, the handle assembly 22 turns freely relative to the coupling socket 30 and tool 12. Like the behavior in the “forward” direction, the free rotation of the handle assembly 22 permits a user to turn the handle assembly 22 in a short arc in direction B to reverse the tool 12 a distance out of the bone, turn the handle assembly 22 in direction A back to the starting point of the short arc, and then turn the handle assembly 22 again in direction B to further reverse the tool 12 from the bone. For both the “forward” and “reverse” directions, the ability of a user to manipulate the tool 12 using an arc of motion less than 360 degrees permits the user to rotate the handle assembly 22 in a range of motion where the user has a higher biomechanical advantage than, for instance, having to awkwardly rotate the handle assembly 22 throughout an entire 360 degrees.

Also present on indicia 28a is an intermediate position of the rotation selector 28 titled “lock,” as shown in FIG. 3. In this position of the rotation selector 28, the handle assembly 22 is fixed against free rotation in either directions A or B relative to coupling socket 30. Thus, the ratchet mechanism 26 of coupling assembly 16 is effectively a solid connection between the coupling socket 30 and the handle assembly 22. In a preferred embodiment, the rotation selector 28 is configured to rotate between three discrete positions corresponding to the “forward,” “lock,” and “reverse” configurations. Rotating the rotation selector 28 would ideally produce tactile and auditory feedback to the user to identify the transition between the different positions. For example, if the user had rotated the rotation selector 28 in direction A before driving the tool 12 into a bone, rotating the selector 28 in direction B to the “reverse” position would produce two clicks as the selector 28 enters the “lock” position and then enters the “reverse” position.

Another preferred feature of the mechanical driver 10 is that the ratchet mechanism 26 provides a ratcheting action when the handle assembly 22 turns freely relative to the coupling socket 30. When the handle assembly 22 is turned opposite the direction of free rotation, the ratchet mechanism 26 engages such that the coupling socket 30 and tool 12 turn with the handle assembly 22. In one form, the ratchet mechanism 26 permits ratcheting increments of less than 20 degrees in the direction of free rotation. This permits a user to make fine incremental adjustments to the rotational position of the handle assembly 22 relative to the coupling socket 30 before rotating the handle assembly 22 in the opposite direction to turn the coupling socket 30 and tool 12 therewith. Additionally, the ratchet mechanism 26 may provide tactile and auditory feedback to the user as the coupling socket 30 rotates freely relative to the handle assembly 22.

To provide the above-described selective rotation and ratcheting ability, those of skill in the art will appreciate a variety of mechanisms may be used, such as a pawl and ratchet or clutch design. A preferred mechanism is disclosed in U.S. Patent Application Publication Number 2006/0248988 to Bennett, which published on Nov. 9, 2006, the entirety of which is incorporated by reference as if fully set forth herein.

Referring now to FIG. 3, socket opening 32 is configured to receive a quarter inch square drive shaft located on tool end 20. However, the socket opening 32 may be configured to receive tool ends having a variety of different shapes, such as Trinkle, A. O., or shapes unique to certain cannulated tool manufacturers. Preferably, the socket opening 32 is configured to restrict relative rotation between the tool end 20 and the coupling socket 30 when the tool end 20 is seated within the socket opening 32. For example, if the tool end 20 is rectangular, the coupling socket 30 may have a complementary rectangular pocket formed within the coupling socket 30. In this manner, the corners formed in the coupling socket 30 would receive the corners of the tool end 20 to restrict the tool end 20 from rotating within the coupling socket 30.

Additionally, the coupling socket 30 includes an internal taper 38 that connects the larger diameter socket opening 32 to a smaller diameter bore 40 which extends through the ratchet mechanism 26. The bore 40 is sized to accommodate the guide wire 14 extending beyond the tool 12 and through the coupling assembly 16. In one form, the coupling socket 30 may be configured so that the tool end 20 seats against the internal taper 38 such that the internal taper 38 restricts movement of the tool end 20 within the coupling socket 30. The operation of the coupling socket 30 and the ratchet mechanism 26 will be discussed in greater detail below.

Turning now to the handle assembly 22, a user may apply compressive or rotational loads to the tool 12 or instruments connected to the tool, such as a bone screw, via the handle assembly 22. In use, gripping portion 24 is utilized to firmly hold the mechanical driver 10. The gripping portion 24 extends away from the ratchet mechanism 26 in a direction generally transverse to the longitudinal axis of bore 40 such that the gripping portion 24 is uninterrupted by the coupling assembly through bore 18. The elongate configuration of the gripping portion 24 provides a levered mechanical advantage to turn the tool 12. In another aspect, the elongate configuration allows the user to hold the mechanical driver 10 while at a safe distance from x-ray radiation that may be used during a surgical procedure. Preferably, handle assembly 22 is made from Radel® brand plastic or another radiolucent polymer that is relatively rigid and provides high impact resistance while permitting sterilization by autoclave.

Four gripping notches 42a, 42b, 42c, and 42d are formed on the gripping portion 24 and are spaced longitudinally along the length of the gripping portion 24. Rounded peaks 44a, 44b, 44c are located between the gripping notches 42 and are spaced apart so that the gripping notches 42 are sized to accommodate a variety of finger sizes. In use, the user's index finger is received within notch 42a, the middle finger within notch 42b, the ring finger within notch 42c, and the little finger within notch 42d.

The handle assembly 22 includes additional features to improve the ergonomic interface between the user's hand and the gripping portion 24. The handle assembly 22 includes left and right sides 46, 48 that are generally flat and extend parallel to one another. Beveled edges 50, 52 are formed at the intersection of each side 46, 48 with the gripping notches 42 and the rounded peaks 44. Similarly, curved surfaces 54, 56 are formed at the intersection of an upper surface 58 of the handle assembly 22 and left and right sides 46, 48. In this manner, a majority of the gripping portion 24 lacks sharp corners which could make holding the handle assembly 22 less comfortable for a user.

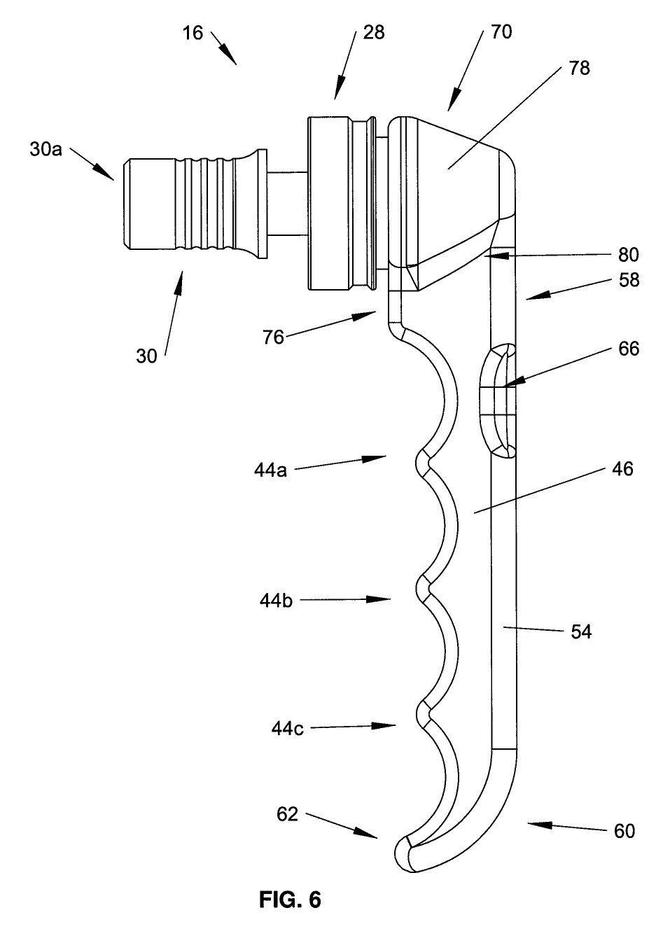

Handle assembly 22 also includes a tapered section 60 that terminates in peak 62, as shown in FIG. 6. Peak 62 is more pronounced than peaks 44a, 44b, and 44c, and tends to restrict movement of a user's hand along the handle assembly 22 during use. This feature is beneficial in that it tends to restrain a user's hand from sliding off of the handle assembly 22 as the handle assembly 22 is being rotated. Referring to FIGS. 1 and 4, the tapered section 60 has a curved surface 64 that seats against the palm of a user's hand. Curved surfaces 54, 56 may extend along the tapered section 60 to further smooth the overall shape of the gripping portion 24. The handle assembly 22 further includes thumb notches 66, 68 formed on opposing sides of the upper surface 58. If the handle assembly 22 is grasped with a right hand, the hand's thumb will be predominantly in contact with the left side 46 and the left thumb notch 66. Conversely, a left-handed grip on handle assembly 22 disposes the hand's thumb predominantly in contact with the right side 48 and the right thumb notch 68.

Referring to FIGS. 4-6, the handle assembly 22 has an end portion 70 that generally surrounds a section of the ratchet mechanism 26. At the end portion 70, the upper surface 58 has an opening 72 wherein a cannulated end 74 of the ratchet mechanism 26 sits flush with the upper surface 58 when the ratchet mechanism 26 is connected to the handle assembly 22. The end portion 70 is generally bounded by the upper surface 58 and a lower surface 76, which are preferably flat and extend parallel to one another. The flat upper surface 58 permits a user to tap the upper surface 58 with a mallet to initially engage the tool 12 into the target bone. Further, the upper end 58 and coupling socket end 30a generally define an overall height of the mechanical driver 10, with the gripping portion 24 extending transverse to the coupling assembly 16 along a length greater than the overall height of the mechanical driver, as shown in FIG. 6.

The end portion 70 also includes a conical surface 78 extending between the upper surface 58 and the lower surface 76 with the conical surface 78 being broader at the juncture with the lower surface 76 than at the juncture with the upper surface 58. As will be discussed in greater detail below, the ratchet mechanism 26 has a larger cross-section at the handle lower surface 76 than at the cannulated end 74 which sits flush with the handle upper surface 58. The conical surface 78 permits the handle assembly 22 to accommodate the larger cross-section of ratchet mechanism 26 while maximizing the surface area of gripping portion 24. In one embodiment, the gripping portion 24 may include the surfaces of the handle assembly 22 generally bounded by left and right sides 46, 48. There, the conical surface 78 permits the left and right sides 46, 48 to extend toward the bore 40 and eventually terminate at point 80, as shown in FIG. 6. This additional gripping surface area provides greater flexibility during surgery by increasing the number of potential hand positions to grasp the handle assembly 22.

In one embodiment, the mechanical driver 10 may include a pin 82 positioned within a bore 84 formed in upper surface 58, as shown in FIG. 4. The pin 82 may be secured within the end portion 70 by epoxy, adhesive, or a threaded engagement. The pin 82 extends into a bore formed in the ratchet mechanism 26 and may be secured therein using a similar approach. In this manner, the pin 82 may restrict rotational movement of the ratchet mechanism 26 within the handle assembly 22. The pin 82 is preferably a safety feature that restricts rotation of the ratchet mechanism 26 within the handle assembly 22 in the event that the primary connection therebetween fails.

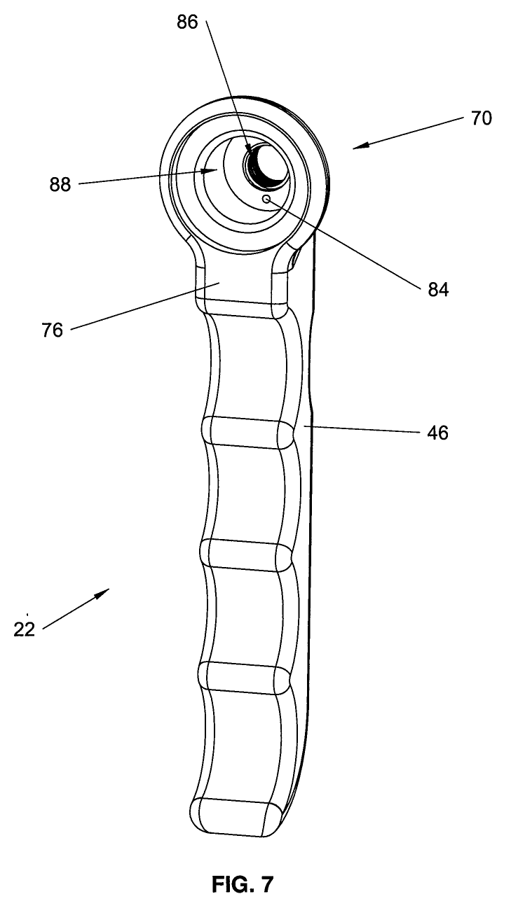

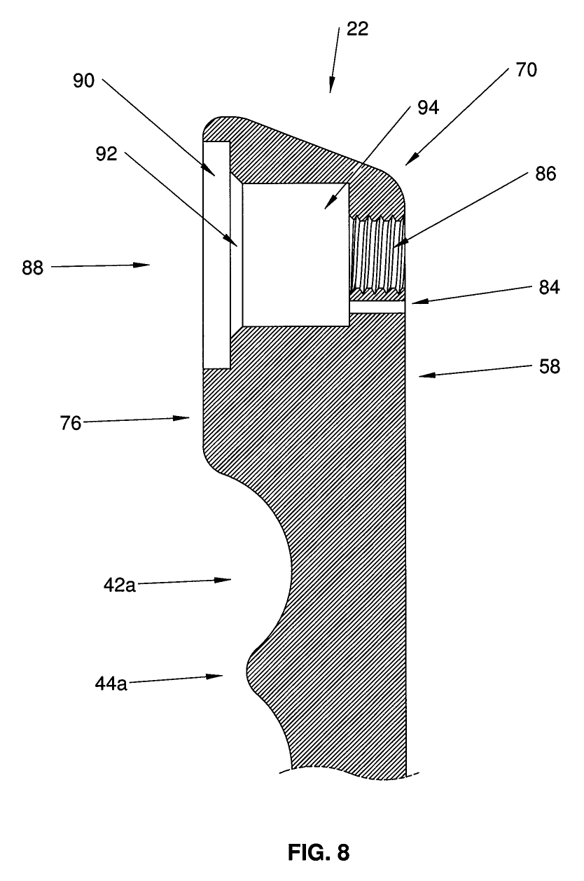

Specifically, the primary connection between the ratchet mechanism 26 and the handle assembly 22 utilizes a threaded bore 86 formed in the end portion 70, as shown in FIGS. 7 and 8. Epoxy is placed onto the threads before the ratchet mechanism 26 and the handle assembly 22 are engaged, so that once the epoxy hardens, the ratchet mechanism 26 and handle assembly 22 are securely joined together. In alternative embodiments, the ratchet mechanism 26 and handle assembly 22 may be joined together using, among other methods, a press fit engagement, one or more fasteners, or adhesives.

The end portion 70 has a through bore 88 between the upper and lower surfaces 58, 76 in which the coupling assembly 16 is attached so that the through bore 18 of the coupling assembly 16 and the through bore 88 of the end portion 70 are coaxial. Because the handle assembly 22 generally extends transverse to the coupling assembly 16, the through bore 88 does not need to be long enough to accommodate the length of the gripping portion 24. This permits the length of the end portion through bore 88 to be shorter than the length of the coupling assembly through bore 18. In this manner, only a short portion of the guide wire 14 extends through the coupling assembly through bore 18. In one form, the length of the end portion through bore 88 is shorter than the length between the socket opening 32 and the upper surface 58 when the coupling assembly 16 is connected to handle assembly 22. The relatively thin profile of the end portion 70 limits obstruction of the length of guide wire 14 extending beyond the tool 12. In this respect, the coupling assembly 16, rather than the handle 10, dictates the length of the obscured portion of the guide wire 14 because the end portion 70 is flush with the cannulated end 74 of the ratchet mechanism 26.

FIG. 8 illustrates a cross-sectional view of the handle assembly 22 with the coupling assembly 16 and the pin 82 removed. The through bore 88 includes a lower section 90 extending through the lower surface 76, and a threaded bore 86 that extends through the upper surface 58. The through bore 88 also includes a transition section 92 and an intermediate section 94. The pin bore 84 is generally in communication with the intermediate section 94 to permit the pin 82 to extend into a bore formed in the ratchet mechanism 26.

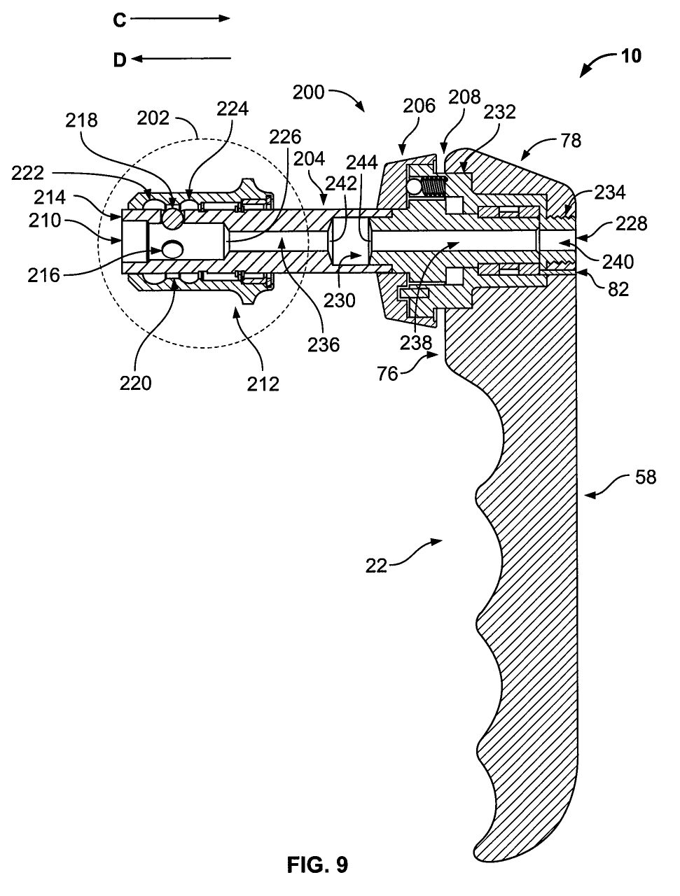

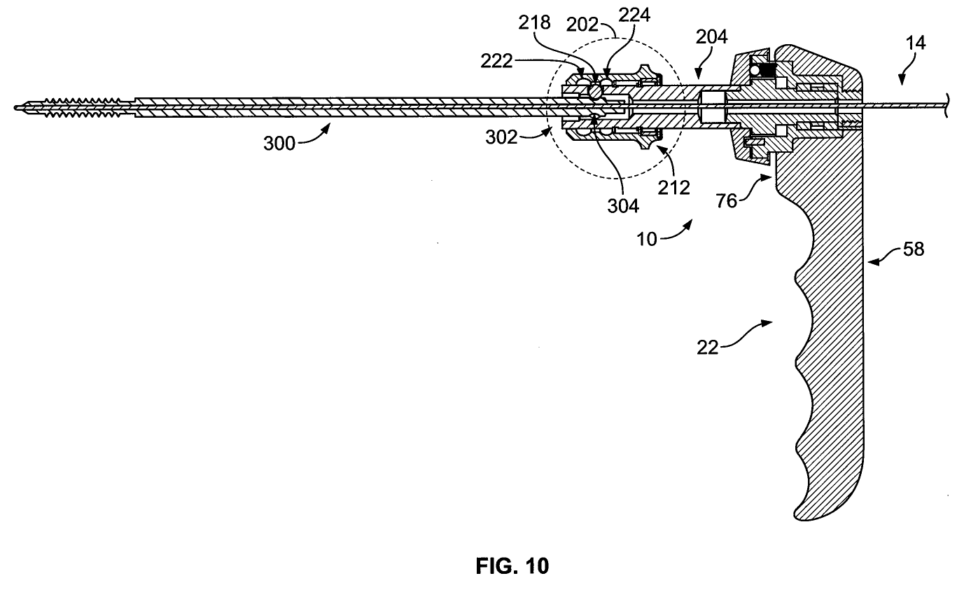

A preferred embodiment of the mechanical driver 10 is shown in FIGS. 9 and 10, wherein the cross-sectional view illustrates the internal features of a coupling assembly 200 secured to handle assembly 22. The coupling assembly 200 is similar to the driving tool disclosed in U.S. Patent Application Publication No. 2006/0248988 to Bennett, which is fully incorporated by reference herein. However, it is within the skill in the art to configure a number of different coupling assemblies with selectively engaging ratchet mechanisms to provide a similar functionality. Generally, the coupling assembly 200 includes a coupling socket 202, a cannulated spindle 204, a rotation selector 206, and a ratchet mechanism 208.

The coupling socket 202 is configured to receive an end 302 of a tool 300, but generally receives a tool end within socket opening 210. The coupling socket 202 is a quick-disconnect coupling that permits rapid locking and unlocking to the tool end 302. The coupling socket 202 may be configured to engage different tool end 302 geometries, for instance a quarter-inch square drive.

More specifically, the coupling socket 202 includes a cylindrical sleeve 212 that is sized to concentrically fit over and slide along a cylindrical tool engaging interface 214 of the coupling socket 202. The sleeve 212 is free to rotate about the interface 214, but is limited to translating a predetermined distance in either the C or D directions. Positioned between the sleeve 212 and the interface 214 is a spring (not shown) that returns the sleeve 212 to an intermediate position as shown in FIG. 9. The tool engaging interface 214 has one or more apertures 216 formed therein with a small shoulder formed at the inner surface of the interface 214 to restrict a ball bearing 218 seated within an aperture 216 from passing fully into the socket opening 210. However, the ball bearing 218 is permitted to partially pass into the opening 210 to engage an annular recess 304 formed in tool end 302 when the coupling socket 202 is in the locked position, as shown in FIG. 10.

The interior surface of the sleeve 212 has a ball locking surface 220 interposed between two ball release recesses 222, 224. When the sleeve 212 is shifted in either the C or D directions, one of the ball release recesses 222, 224 is aligned with the ball bearing 218 so that the ball bearing 218 may partially pass into the sleeve 212. In this manner, the ball bearing 218 is free to shift out of the way of the tool end 302 when the tool end 302 is placed into tool engaging interface 214. To lock the coupling assembly 200 to the tool 300, the sleeve 212 is released which allows the spring to return the sleeve 212 to the intermediate position such that the ball locking surface 220 is positioned over the ball bearing 218. Because the ball bearing 218 is restricted from shifting into the sleeve 212, the ball bearing 218 sits within the recess 304 on the tool end 302. To unlock the coupling assembly 200 from the tool 300, the sleeve is again shifted in either the C or D directions to position one of the ball release recesses 222, 224 over the ball bearing 218 so that the ball bearing 218 may again pass into the sleeve 212. Then, the tool 300 is pulled out of the coupling socket 302 which causes the geometry of tool end 302 to shift the ball bearing 218 into the sleeve 212. For tool ends 302 without a recess 304, a differently shaped element may be used instead of the ball bearing 218 to engage the tool end 302, such as a cylinder or a tapered element.

When the tool end 302 is positioned within the tool engaging interface 214, the tool end 302 may be spaced from an internal taper 226 which separates the larger diameter socket opening 210 from a smaller diameter bore 228 of the coupling assembly 200. In an alternative design, the tool end 302 may be configured to extend the full length of the opening 210 and be seated against the internal taper 226. As shown in FIG. 9, the smaller diameter bore 228 may extend along several interconnected elements including a toothed hub 230 of the ratchet mechanism 208 that rotates within a housing 232. In a preferred form, the housing 232 extends from the lower surface 76 to the upper surface 58 of the handle assembly 22. The housing 232 is flush with the end of the through bore 88 at upper surface 58 and extends beyond the other end of the through bore 88 for being connected to the rest of the coupling assembly 16. Further, the housing 232 has threads 234 that engage the threaded bore 86 to fixedly engage the housing 232 to the handle assembly 22 in coaxial alignment with the through bore 88 of the handle assembly 22.

Within the housing 232 are a pair of pawls (not shown) that control rotation of the toothed hub 230 relative to the handle assembly 22. The rotation selector 206 is connected to the housing 232 so that a user may rotate the rotation selector 206 to adjust the positioning of the pawls and thereby control how the toothed hub 230 rotates within the housing 232. In the illustrated form, the cannulated spindle 204 is a separate element that is connected to the toothed hub 230. In an alternative embodiment, the spindle 204 and toothed hub 230 may be integrally formed. Similarly, although smaller diameter bore 228 generally includes a spindle bore 236, a hub bore 238, and a housing bore 240, an integral construction of the spindle 204 and toothed hub 230 would have a more uniform profile along the length of smaller diameter bore 228. The bores 236, 238, 240 are coaxially aligned which improves the ease with which the guide wire 14 may be passed through the coupling assembly 200. Additionally, tapered sections 226, 242, 244 may be positioned within the coupling assembly 200 to direct the guide wire 14 as the guide wire 14 passes between the different bores.

Once the ball bearing 218 is seated within the recess 304, the coupling socket 202 is locked onto the tool 300. The tool engaging interface 214 is configured to resist rotation of the tool 300 relative to the coupling socket 202. For example, if the tool end 302 is a square drive, the tool engaging interface 214 may have a rectangular seat (not shown) for receiving the tool end 302. Referring again to FIG. 10, the mechanical driver 10 and tool 300 may be placed over the guide wire 14 and slid therealong until the tool 300 reaches the end of the guide wire 14. As is apparent, the guide wire 14 projects from the tool end 302 and along the interior of coupling assembly 200 until reaching the upper surface 58 of the handle assembly 22, at which point the guide wire 14 extends beyond the handle assembly 22.

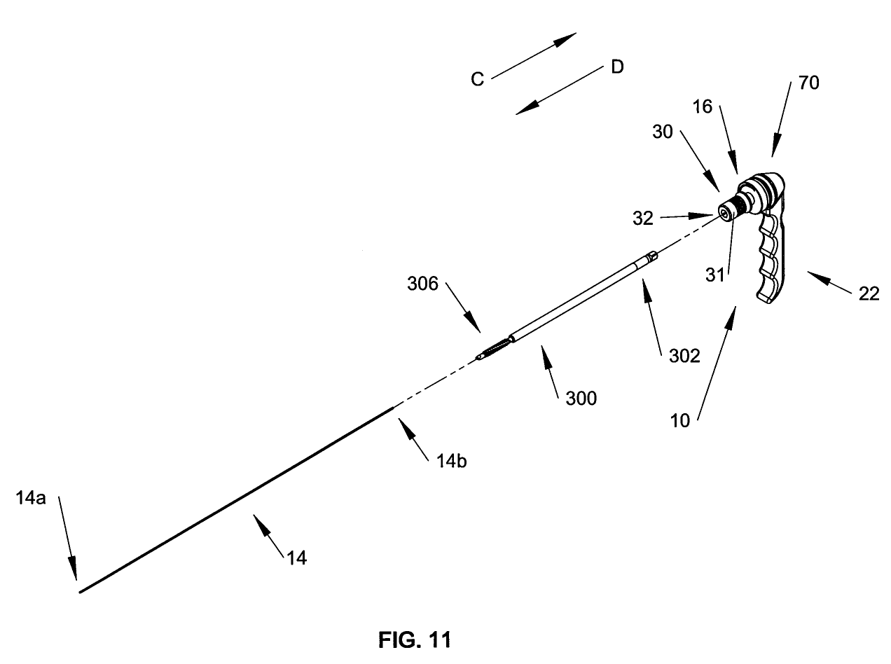

Referring to FIGS. 11-13, an exemplary method of using an embodiment of the mechanical driver 10 to prepare the surface of a bone with tool 300. Using a Jamshidi needle or other tool, a first end 14a of the guide wire 14 is positioned on the bone surface and the guide wire 14 is generally located along the desired trajectory for the bone screw into the bone. To secure the guide wire 14 to the bone, the guide wire 14 is lightly tapped with a hammer at second end 14b.

The mechanical driver 10 is then releasably connected to the tool 300. In a manner similar to the embodiments of FIGS. 9 and 10, the handle 10 includes a sleeve 31 that is shifted from an intermediate position in either the C or D directions to configure the coupling socket 30 to receive tool end 302. Once the tool end 302 is located within the socket opening 32, the sleeve 31 is shifted back to the intermediate position to lock the coupling socket 30 onto the tool 300. At this point, the tool 300 is fixed relative to the coupling socket 30 such that the tool 300 is restrained against linear or rotational movement relative to the coupling socket 30.

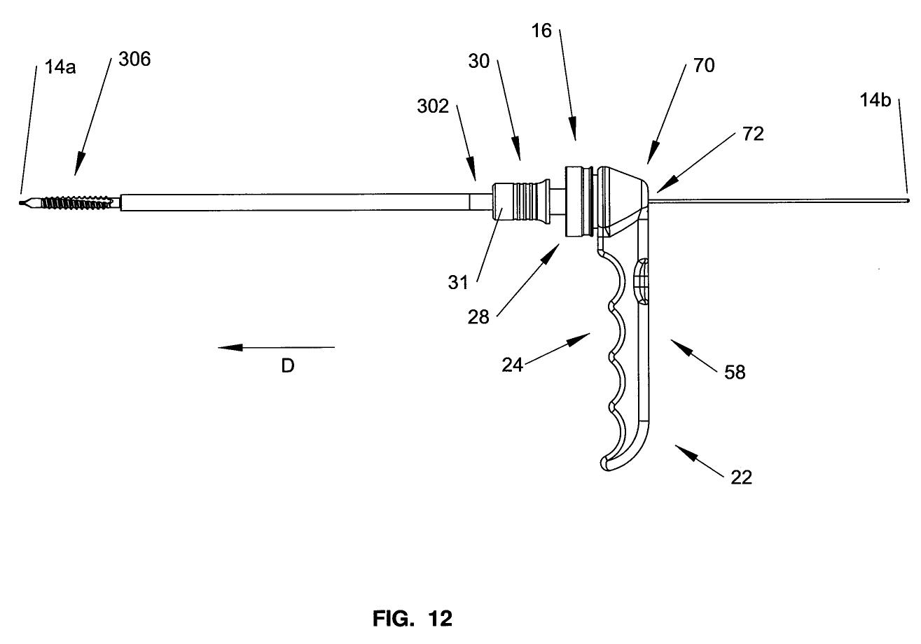

Next, the tool 300 is placed onto guide wire 14 by inserting the second end 14b of the guide wire into the cannula of an engagement end 306 of the tool 300. The tool 300 and mechanical driver 10 are slid along the guide wire 14 until the tool 300 reaches the guide wire first end 14a, as shown in FIG. 12. Concurrently, the second guide wire end 14b travels along the cannulated interior of the tool 300 before projecting outward from the tool end 302. The cannulated features of coupling assembly 16 permit the second end 14b to pass from the coupling socket 30 to the opening 72 formed in the end portion 70 of handle assembly 22. With the handle assembly 22 extending transversely to the coupling assembly 16, the gripping portion 24 is positioned out of the path of the second guide wire end 14b as it reaches and projects through the opening 72. In this manner, the gripping portion 24 may be grasped to guide the tool 300 along the guide wire 14 without placing the user's hand in the path of the guide wire 14.

Once the tool engagement end 306 is in contact with the bone surface, a mallet may be used to initially drive the engagement end 306 into the bone. In one approach, the user taps the upper surface 58 with the mallet to apply a compressive force in direction D as shown in FIG. 12. The compressive force drives the tip of engagement end 306 into the bone. The flat and relatively long upper surface 58 provides a large, uninterrupted striking surface for the user's mallet which reduces the likelihood of uncontrolled glancing blows. Preferably, the engagement end 306 is driven into the bone only as deep as necessary to permit the cutting surfaces of the engagement end 306 to engage the bone when the tool 300 is rotated. In some instances, a mallet may be unnecessary such that the user need only apply a compressive force in direction D and rotate the tool 300 to engage the tool cutting surfaces with the bone.

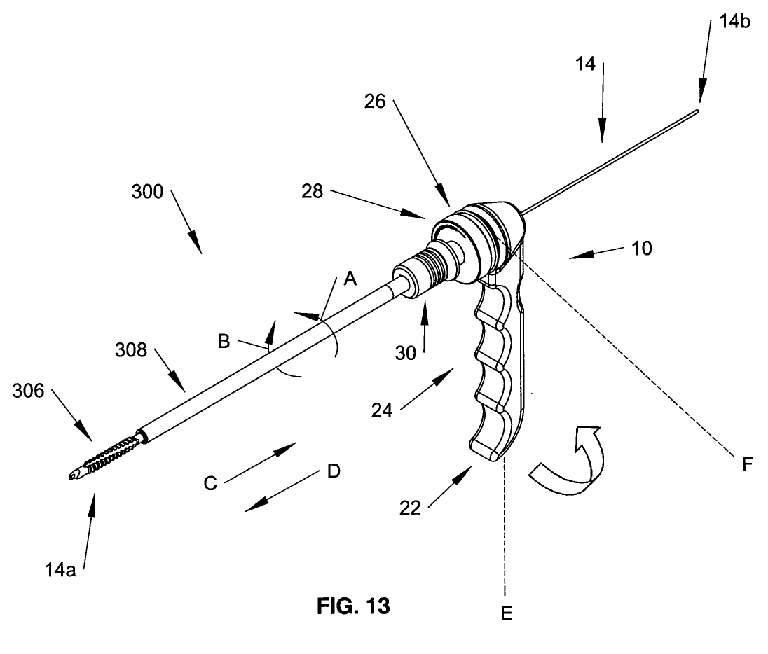

The rotation selector 28 is then rotated to restrict rotation of the tool 300 relative to the handle assembly 22 and permit the tool 300 to be rotatably driven into the bone. In one embodiment, tool 300 is configured so that rotation of the tool 300 in direction A about an axis generally defined by the guide wire 14 will tend to engage tool cutting surfaces with the bone, as shown in FIG. 13. Conversely, rotation in direction B will tend to disengage the cutting surfaces from the bone. Given this configuration, the rotation selector 28 is rotated in direction A so that ratchet mechanism 26 fixes coupling socket 30 relative to the handle assembly 22 when the handle assembly 22 is turned in direction A. Though turning the handle assembly 22 in direction A will turn the coupling socket 30 and tool 300 therewith, the ratchet mechanism 26 permits the handle assembly 22 to turn relative to the coupling socket 30 and tool 300 in direction B. Alternatively, if rotation of the tool 300 in the B direction tended to engage the tool cutting edges with the bone, the rotation selector 28 would be rotated in direction B before driving the tool 300 into the bone.

With the mechanical driver 10 ready to turn the tool 300, the user may brace the tool shaft 308 with one hand before grasping the gripping portion 24 with the other hand and turning handle assembly 22 in direction A. Preferably, the hand bracing the tool shaft 308 grasps the tool shaft 308 in a manner that permits rotation of the tool shaft 308 but allows the user to firmly hold on to the mechanical driver 10. For exemplary purposes, FIG. 13 shows handle assembly 22 initially extending along an axis E. Then, the handle assembly 22 is turned in direction A about the guide wire 14 to drive the tool 300 into the bone until the handle assembly 22 extends along an axis F. At this position, the user may stop turning the handle assembly 22 in direction A and reverse rotation so that the handle assembly 22 returns to alignment with axis E. Because the rotation selector 28 and ratchet mechanism 26 were configured to permit the handle assembly 22 to turn relative to the coupling socket 30 in direction B, the turning of handle assembly 22 back to axis E does not produce concurrent turning of the tool 300. Further, the engagement of the tool 300 within the bone tends to resist turning of the tool 300 and connected coupling 30 so the user may reposition the handle assembly 22 before again driving the tool 300 deeper into the bone. Although the range of motion between axes E and F is arbitrary for exemplary purposes, the mechanical driver 10 permits a user to select a range of motion that is relatively short but provides the maximum biomechanical advantage to turn the tool 300.

As is apparent, the transversely extending handle assembly 22 provides a levered mechanical advantage for turning the tool 300 when the user grasps the gripping portion 24. Specifically, the transversely extending handle assembly 22 acts as a lever arm relative to the tool 300. When the user applies a force against the gripping portion 24, the handle assembly 22 multiplies this force and generates a torque about the tool 300. The amount of torque may be increased by grasping the gripping portion 24 farther away from the tool 300 to effectively increase the levered mechanical advantage with which the user is turning the tool 300.

When the handle assembly 22 is turned, the gripping portion 24 turns about the tool 300 in an arcuate path spaced from the tool 300 so that the gripping portion 24 is positioned out of the path of the guide wire 14 as the tool 300 is driven into the bone. Additionally, the gripping notches 42a, 42b, 42c, 42d and thumb notches 66, 68 enhance the ergonomic fit between the user's hand and the gripping portion 24 such that a user naturally tends to grasp the handle assembly 22 in a manner that provides maximum control over the mechanical driver 10 and the tool 300. Although the user's index through little fingers are generally received respectively within gripping notches 42a, 42b, 42c, 42d, the handle assembly 22 permits a variety of alternative hand positions to turn the handle 300.

For example, one method of rapidly driving the tool 300 into the bone involves positioning the user's thumb on the left side 46 of the handle assembly 22 and the index through little fingers on the right side 48 of the handle assembly 22. Using only the user's fingertips to grasp the handle assembly 22, the handle assembly 22 is turned from axis E to axis F to drive the tool 300 into the bone. The handle assembly 22 is quickly returned to alignment with axis E using a flicking motion of the user's wrist, before the handle assembly 22 is again turned from axis E to axis F to drive the tool 300 deeper into the bone. By repeating this method in a rapid fashion, the tool 300 is quickly driven into the bone.

Once the tool 300 has reached the correct depth within the bone, the rotation selector 28 is rotated in direction B to reverse the direction in which the tool 300 turns with the handle assembly 22. Then, turning the handle assembly 22 in direction B will turn the tool 300 therewith to disengage the tool 300 from the bone, while the handle assembly 22 may turn freely relative to the tool 300 in direction A. In a manner similar to the procedure for engaging the tool 300, the tool 300 may be disengaged from the bone using a short arc of motion that removes the tool 300 as the handle assembly 22 turns in direction B but keeps the tool 300 stationary as the handle assembly 22 returns in direction A. Alternatively, a rapid removal of the tool 300 may be accomplished using the wrist-flicking procedure outlined above.

Upon removal of the tool 300 from the bone, the mechanical driver 10 may be disengaged from the tool 300 by shifting the sleeve 31 in direction C or D and removing the tool end 302 from the coupling socket 30. The mechanical driver 10 may now be connected to a different tool that is to be guided into contact with the bone using the guide wire. For example, the mechanical driver 10 may be connected to a cannulated screw inserter for inserting a bone screw into the bone. Although the mechanical driver 10 is particularly well-suited for use with cannulated rotatable tools, the mechanical driver 10 may also be used with non-cannulated tools, or even tools that engage a bone surface in a linear manner without rotation.

While there have been illustrated and described particular embodiments of the present invention, it will be appreciated that numerous changes and modifications will occur to those skilled in the art, and it is intended in the appended claims to cover all those changes and modifications which fall within the true spirit and scope of the present invention.

What is claimed is:

1. A device for rotatably driving a cannulated tool along a guide wire, the device comprising:
an elongate coupling assembly having an axial through bore in which the tool and the guide wire extending through the tool are received;
a handle assembly having an end portion connected to the coupling assembly for rotatably driving the tool;
a ratchet mechanism between the handle assembly and the coupling assembly configured so that turning the handle assembly in one rotary direction turns the coupling assembly therewith and the handle assembly turns relative to the coupling assembly in an opposite rotary direction; and
an elongate gripping portion of the handle assembly that extends transversely to the elongate coupling assembly from the handle assembly end portion to provide a levered mechanical advantage for turning the coupling assembly with a user gripping the handle assembly gripping portion.

2. The device of claim 1 wherein the coupling assembly has a predetermined length along the longitudinal axis thereof, and the gripping portion of the handle assembly at the end portion thereof has upper and lower surfaces spaced from each other along the longitudinal axis by a distance that is less than the predetermined length of the coupling assembly.

3. The device of claim 1 wherein the handle assembly at the end portion thereof has a through bore extending therethrough in coaxial alignment with the through bore of the coupling assembly, and the handle assembly includes a housing for the ratchet mechanism that is fixed in the handle assembly through bore to extend therethrough for the full axial extent thereof.

4. The device of claim 3 wherein the handle assembly through bore has opposite ends and the ratchet mechanism housing is flush with one end of the through bore and extends beyond the other end thereof for being connected to the coupling assembly.

5. The device of claim 1 wherein the elongate gripping portion includes a plurality of gripping notches spaced longitudinally along the gripping portion.

6. The device of claim 1 wherein the elongate gripping portion includes an upper surface and thumb notches formed on opposing sides of the upper surface for both left- and right-handed usage of the device.

7. The device of claim 1 wherein the elongate coupling assembly includes a quick disconnect coupling for engaging the tool.

8. The device of claim 1 wherein an upper surface of the handle assembly and an end of the elongate coupling assembly generally define an overall height of the device along the longitudinal axis of the elongate coupling assembly and the elongate gripping portion extends transverse to the coupling assembly along a length greater than the overall height of the device.

9. A mechanical driver for rotatably driving a cannulated tool, the mechanical driver comprising:
a coupling socket having a socket opening for removably receiving an end of the cannulated tool therein;
a transverse handle for being gripped to rotate the coupling socket and tool;
a cannulated connection assembly between the socket and transverse handle having an elongate through bore extending along a longitudinal axis thereof and in communication at one end with the socket opening;
upper and lower surfaces of the transverse handle that extend transversely to the through bore along the handle;
a coupling end portion of the handle having a through bore between the upper and lower surfaces of the handle in which the connection assembly is attached so that the through bores are coaxial, the through bore of the handle coupling end portion being shorter than a predetermined length of the connection assembly through bore extending from the one end thereof to the upper surface of the handle coupling end portion so that only a short portion of a guide wire extends through the connection assembly through bore.

10. The mechanical driver of claim 9 wherein the transverse handle includes an elongate gripping portion extending away from the coupling end portion of the handle with the gripping portion being uninterrupted by the through bore extending between the upper and lower surfaces of the handle.

11. The mechanical driver of claim 9 wherein the cannulated connection assembly includes a drive reverser configured so that turning the transverse handle in one rotary direction turns the coupling socket therewith and the transverse handle turns relative to the coupling socket in an opposite rotary direction.

12. The mechanical driver of claim 9 wherein the transverse handle includes an elongate portion extending radially outward from the longitudinal axis of the cannulated connection assembly with a gripping surface disposed thereon so that the entirety of the gripping surface rotates in an arc about the cannulated connection assembly as the tool is rotatably driven.

13. The mechanical driver of claim 9 wherein the coupling end portion of the handle includes a through bore formed in the upper surface of the handle and a pin positioned within the through bore that extends into a bore formed in the connection assembly to restrict relative rotational movement between the handle and a portion of the connection assembly.

14. The mechanical driver of claim 9 wherein the coupling end portion of the handle includes an outer surface with a conical shape extending between the upper and lower surfaces with the outer surface being wider at the juncture with the lower surface than at the upper surface.

15. The mechanical driver of claim 9 wherein the transverse handle includes an elongate gripping portion extending away from the coupling end portion of the handle and a peak positioned opposite the coupling end portion along the handle, the peak extending transverse to the elongate gripping portion between the upper and lower surfaces of the transverse handle to restrain a user's hand from sliding off the handle as the handle is being rotated.

16. A method of driving an elongate, cannulated tool along a guide wire and into a bone, the method comprising:
releasably connecting a handle assembly to the tool;
positioning the tool adjacent the surface of the bone; and
turning a gripping portion of the handle assembly about the tool in an arcuate path spaced from the tool so that the gripping portion is positioned out of the path of the guide wire as the tool is rotatably driven into the bone.

17. The method of claim 16 including placing the guide wire a predetermined distance into the bone and translating the guide wire relative to the tool and the handle assembly at a distance spaced from the gripping portion as the tool is driven into the bone.

18. The method of claim 16 including passing a portion of the guide wire beyond the handle assembly along a path spaced from the gripping portion and maintaining the length of the guide wire portion projecting beyond the handle assembly as the tool is driven into the bone so that a surgeon may observe whether the guide wire has bound to the tool.

19. The method of claim 16 including turning the gripping portion in a reverse direction without turning the tool and turning the gripping portion in the original direction to drive the tool further into the bone using an arc of motion less than 360 degrees.

20. The method of claim 16 including grasping the gripping portion generally along an axis transverse to the length of the tool and rotating a selector on the handle assembly about the tool to restrict relative rotation between the handle assembly and the tool.

21. The method of claim 16 including grasping the gripping portion to rotate the handle assembly and driving the tool into the bone with greater force by grasping the gripping portion at a greater distance away from the tool.
